(19) United States
(12) Patent Application Publication
(10) Pub. No.: US 2009/0270999 A1
(43) Pub. Date: Oct. 29, 2009

(54) PATCH FOR ENDOSCOPIC REPAIR OF HERNIAS

(76) Inventor: Roderick B. Brown, Glenwood, MN (US)

Correspondence Address: NIKOLAI & MERSEREAU, P.A., 900 SECOND AVENUE SOUTH, SUITE 820, MINNEAPOLIS, MN, 55402, US

(21) Appl. No.: 12/108,876

(22) Filed: Apr. 24, 2008

(51) Int. Cl. A61F 2/02; A61B 17/08

(52) U.S. Cl. 623/23.72; 606/151

(57) ABSTRACT

In some embodiments, a method of repairing a hernia may include one or more of the following steps: (a) introducing a patch prosthesis into a patient, (b) positioning the patch prosthesis over the hernia, (c) removing a wire frame from the patch prosthesis and the patient, (d) wrapping the patch prosthesis, (e) allowing the patch prosthesis to unwrap, (f) removing a removable stitch coupling the wire frame and a prosthetic fabric, and (g) securing the prosthetic fabric to the patient.

### BACKGROUND OF THE INVENTION

I. Field of the Invention

Embodiments of the present invention relate to hernias. Particularly, embodiments of the present invention relate to hernia repair surgery. More particularly, embodiments of the present invention relate to a compressible rolled or folded prosthetic to allow deployment through a laparoscopic cannula or similar device.

II. Discussion of Related Art

A hernia is a protrusion of a tissue, structure, or part of an organ through the muscular tissue or the membrane by which it is normally contained. The hernia has three parts: the orifice through which it herniates, the hernial sac, and its contents. A hernia may be likened to a failure in the sidewall of a pneumatic tire. The tire's inner tube behaves like the organ and the side wall like the body cavity wall providing the restraint. A weakness in the sidewall allows a bulge to develop, which can become a split, allowing the inner tube to protrude, and leading to the eventual failure of the tire.

By far most hernias develop in the abdomen, when a weakness in the abdominal wall evolves into a localized hole, or “defect”, through which adipose tissue, or abdominal organs covered with peritoneum, may protrude. Another common hernia involves the intervertebral disc, and causes back pain or sciatica. Hernias may be present either with pain at the site, a visible or palpable lump, or in some cases by more vague symptoms resulting from pressure on an organ which has become “stuck” in the hernia, sometimes leading to organ dysfunction. Fatty tissue usually enters a hernia first, but it may be followed by or accompanied by an organ. Most of the time, hernias develop when pressure in the compartment of the residing organ is increased, and the boundary is weak or weakened.

It is generally advisable to repair hernias in a timely fashion, in order to prevent complications such as organ dysfunction, gangrene, and multiple organ dysfunction syndrome. Most abdominal hernias can be surgically repaired, and recovery rarely requires long-term changes in lifestyle. Uncomplicated hernias are principally repaired by pushing back, or “reducing”, the herniated tissue, and then mending the weakness in muscle tissue (an operation called herniorrhaphy). If complications have occurred, the surgeon will check the viability of the herniated organ, and resect it if necessary. Modern muscle reinforcement techniques involve synthetic materials avoiding over-stretching of already weakened tissue (as in older, but still useful methods). The mesh is placed over the defect, and sometimes staples, spiral tacks or sutures are used to keep the mesh in place. Evidence suggests this method has the lowest percentage of recurrences and the fastest recovery period. Increasingly, some repairs are performed using laparoscopic techniques.

Many patients are managed through day surgery centers, and are able to return to work within a week or two, while heavy activities are prohibited for a longer period. Patients who have their hernias repaired with mesh often recover in a number of days. Generally, the use of external devices to maintain reduction of the hernia without repairing the underlying defect (such as hernia trusses, trunks, belts, etc.) is not advised. Exceptions are uncomplicated incisional hernias arising shortly after the operation (should only be operated on after a few months), or inoperable patients.

Implantable mesh patches for the repair of inguinal and other abdominal wall hernias are well known in the prior art. Almost all repairs done today are open “tension-free” repairs involving the placement of a synthetic mesh to strengthen the inguinal region; some popular techniques include the Lichtenstein repair (flat mesh patch placed on top of the defect), Plug and Patch (mesh plug placed in the defect and covered by a Lichtenstein-type patch), Kugel (mesh device placed behind the defect), and Prolene Hernia System (two-layer mesh device placed over and behind the defect). This operation is called a ‘hernioplasty’.

The meshes used are typically made from polypropylene or polyester, although some companies market Teflon meshes and partially absorbable meshes or biomaterials. The operation may be performed under local, regional, or general anesthesia, and patients often go home within a few hours of surgery, frequently requiring minimal analgesic medication for post-operative pain. Patients are encouraged to walk and move around immediately after the operation, and can usually resume all their normal activities within a week or two of operation. Recurrence rates are very low—one percent or less, compared with over 10% for a tension repair.

Typically these patches are intended for permanent placement within a patient's body space. For example, prosthesis for use in hernia repair surgery having a preformed prosthetic fabric supported along its periphery by shape memory alloy wire having a transformation temperature corresponding to normal body temperature, allowing the prosthesis to be tightly rolled into a cylindrical configuration for delivery is known.

Laparoscopic surgery has proven to be a preferred surgical technique for addressing inguinal hernias. Facilitated laparoscopic procedures provide a hernia repair patch supported by a single strand of wire Nitinol frame. The patch could be rolled up or folded and inserted into a cannula and then deployed through the cannula into the body to cover the direct, indirect, and femoral hernia space for inguinal hernias, or the defect from an incisional, ventral or umbilical hernia. Because the frame is integral to the patch, it does not migrate and need not be sutured or stapled in place.

It has been found; however, smaller sized cannulas are often preferred in laparoscopic procedures. Patients find trocars with a smaller diameter are less invasive and less painful. A need, therefore, exists for a prefabricated hernia patch to be used in laparoscopic surgery to conform to anatomical structures, readily deployable when released from a tubular laparoscopic introducer, remaining in place without a need for stapling or suturing to the underlying fascia, and which is flexible enough to be rolled or folded to fit into a trocar of a smaller diameter.

### SUMMARY OF THE INVENTION

In some embodiments, a hernia patch may include one or more of the following features: (a) a frame formed from a shape memory alloy wherein the frame has an expanded shape when the shape memory alloy is in a austenite form and a rolled or folded compact shape when in a martensite form, and (b) a prosthetic fabric material attached to the frame by a removable stitch.

In some embodiments, a hernia patch for laparoscopic delivery may include one or more of the following features: (a) a wire frame, (b) a prosthetic fabric, and (c) a removable stitch which couples the wire frame to the prosthetic fabric

In some embodiments, a method of repairing a hernia may include one or more of the following steps: (a) introducing a patch prosthesis into a patient, (b) positioning the patch prosthesis over the hernia, (c) removing a wire frame from the patch prosthesis and the patient, (d) wrapping the patch prosthesis, (e) allowing the patch prosthesis to unwrap, (f) removing a removable stitch coupling the wire frame and a prosthetic fabric, and (g) securing the prosthetic fabric to the patient.

In some embodiments, a method of manufacturing a patch prosthetic may include one or more of the following steps: (a) forming a wire frame, (b) placing a prosthetic fabric adjacent to the wire frame, (c) securing the wire frame to the prosthetic fabric with a removable stitch, and (d) tying off the removable stitch.

### DESCRIPTION OF DRAWINGS

FIG. 1 shows an enlarged plan view of a hernia repair patch constructed in accordance with embodiments of the present invention;

FIG. 2 shows a process diagram for the deployment of a hernia repair patch in an embodiment of the present invention;

FIG. 3 shows a rolled or folded tubular configuration for endoscopic delivery through a trocar in embodiments of the present invention;

FIG. 4 is an end view of the rolled or folded patch of FIG. 3;

FIG. 5 is a perspective view of a heat set mold used to form the cable frame in embodiments of the present invention;

FIG. 6 is an exploded view of a hernia repair patch in an embodiment of the present invention; and

FIG. 7 is a process diagram detailing the construction of a hernia repair patch in an embodiment of the present invention.

### DESCRIPTION OF PREFERRED EMBODIMENT

The following discussion is presented to enable a person skilled in the art to make and use the present teachings. Various modifications to the illustrated embodiments will be readily apparent to those skilled in the art, and the generic principles herein may be applied to other embodiments and applications without departing from the present teachings. Thus, the present teachings are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of the present teachings. Skilled artisans will recognize the examples provided herein have many useful alternatives and fall within the scope of the present teachings.

Referring to FIG. 1, there is illustrated an enlarged view of a hernia repair patch constructed in accordance with embodiments of the present invention. The patch prosthesis is indicated generally by numeral 10 and is seen to include a removable outer wire frame 12 comprising a wire formed from a shape metal alloy, possibly a nickel titanium alloy comprising 49-51 atomic percent nickel and the remainder titanium. Such an alloy is commonly referred to as NiTiNOL™. By proper adjustment of the relative concentration of nickel in the alloy, wire frame 12 can be made to exhibit a transition temperature between the austenite form and the martensite form at about 37° C. corresponding to body temperature. Wire frame 12 is shown as comprising a closed loop which is generally oval in shape.

Supported within wire frame 12 is a prosthetic fabric 14, possibly woven strands of polypropylene plastic, expanded PTFE (Polytetrafluoroethylene), polyester, or a suitable biomaterial. As such, patch prosthesis 10 may be steam sterilized. Sewn to prosthetic fabric 14 and wire frame 12 is a removable stitch 16 which temporarily secures prosthetic fabric 14 to wire frame 12. The operation of removable stitch 16 is discussed in greater detail below.

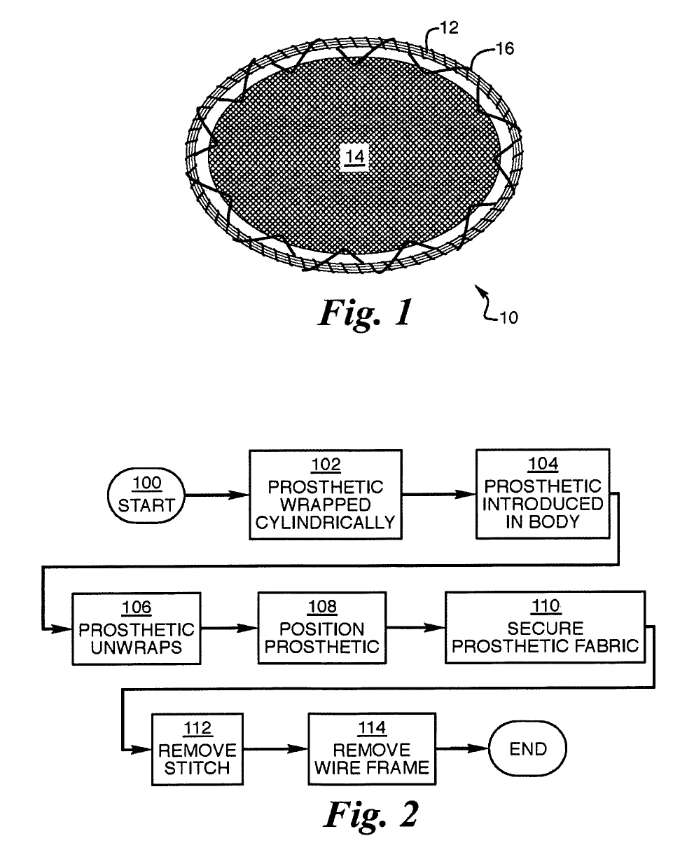

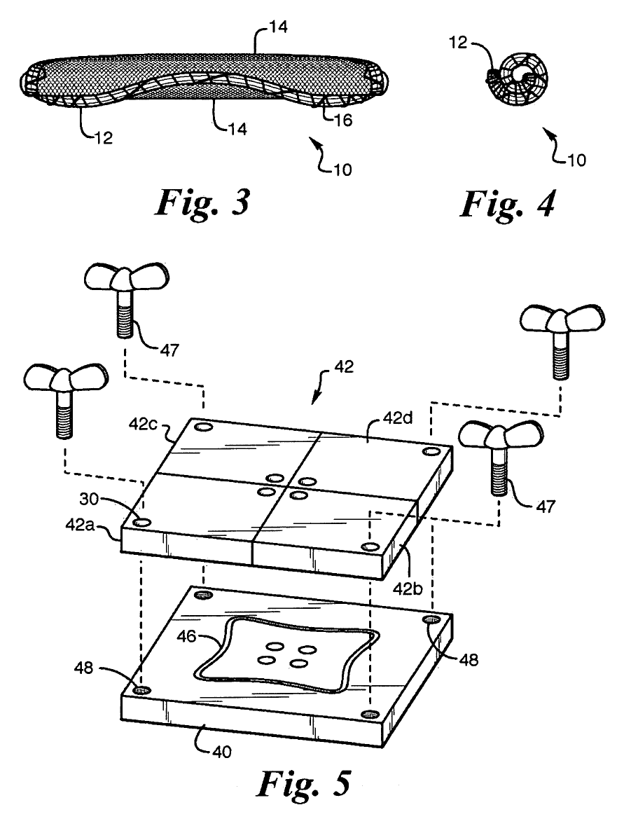

With reference to FIG. 2, a process diagram for the deployment of a hernia repair patch in an embodiment of the present invention is shown. When wire frame 12 is cooled below the transformation state of the shape memory alloy so it is in its martensite form, patch prosthesis 10 can be helically wrapped or folded to form a somewhat cylindrical structure as illustrated in FIGS. 3 and 4 at state 102. This allows patch prosthesis 10 to be introduced into the abdominal cavity through a tubular trocar at state 104. As the shape memory wire frame 12 warms up to body temperature, it transforms to its austenite form as depicted in FIG. 1 at state 106. Using a laparoscopic forceps, patch prosthesis 10 can be grasped and repositioned by the surgeon until appropriately located for covering the hernia defect at state 108. Once the mesh has been positioned, and anchored to the adjacent tissues, at state 110, the surgeon can cut removable stitch 16, grasp a severed end, and remove removable stitch 16 at state 112. Once removable stitch 16 is removed, wire or cable frame 12 and prosthetic fabric 14 are no longer joined and wire or cable frame 12 can be removed at state 114.

Those skilled in the art will appreciate the prosthesis may be manufactured in a variety of shapes and sizes to accommodate children, adults, males, females, and especially the type of hernia encountered. It can be contained in a sterile pack until ready for use. While NiTiNOL™ is a good shape memory alloy, other alloys, such as gold-cadmium nickel-aluminum, and manganese-copper would also be suitable. Moreover, prosthetic fabric 14 need not be polypropylene mesh, but can also comprise other suitable materials, such as body-compatible biaxially oriented polymeric films or suitable biomaterials.

Without limitation, the major axis of patch prosthesis 10 may be in a range of from about 8 to 40 cm and the constricted minor axis may be about 6 to 30 cm. Patch prosthesis 10 can be tightly rolled or folded into a cylinder, as shown in FIG. 4, so as to fit within the internal lumen of a trocar or introducer sheath.

FIG. 5 shows a mold structure for use in establishing the desired frame shape to the wire or cable frame 12. It comprises a base plate 40 having a recess 46 formed therein. Recess 46 defines the desired shape configuration for wire frame 12. Wire frame 12 is then fitted piecewise into recess 46 so the cable or wire follows the perimeter of the recess 46. Metal cover plate 42 comprising segments 42a-42d is then affixed to the base plate 40 in pieces as the cable is being forced into recess 46 to prevent wire frame 12 from escaping recess 46. Segments 42a-42d comprising cover plate 42 is then secured to base plate 40 by passing fasteners 47 through cooperating threaded bores 30, 48 in cover plate 42 and base plate 40, respectively. Cover plate 42 is preferably formed from plural segments, thereby allowing piece wise insertion of wire frame 12 into groove or recess 46.

Once inserted, the assembly is then subjected to a heating step for a time and at a temperature imparts a set to the closed loop. After the assembly is sufficiently cooled, top plate segments 42a-42d are unscrewed and wire frame 12 is removed from recess 46. Prosthetic fabric 14 can then be affixed to closed wire frame 12 as discussed in more detail below.

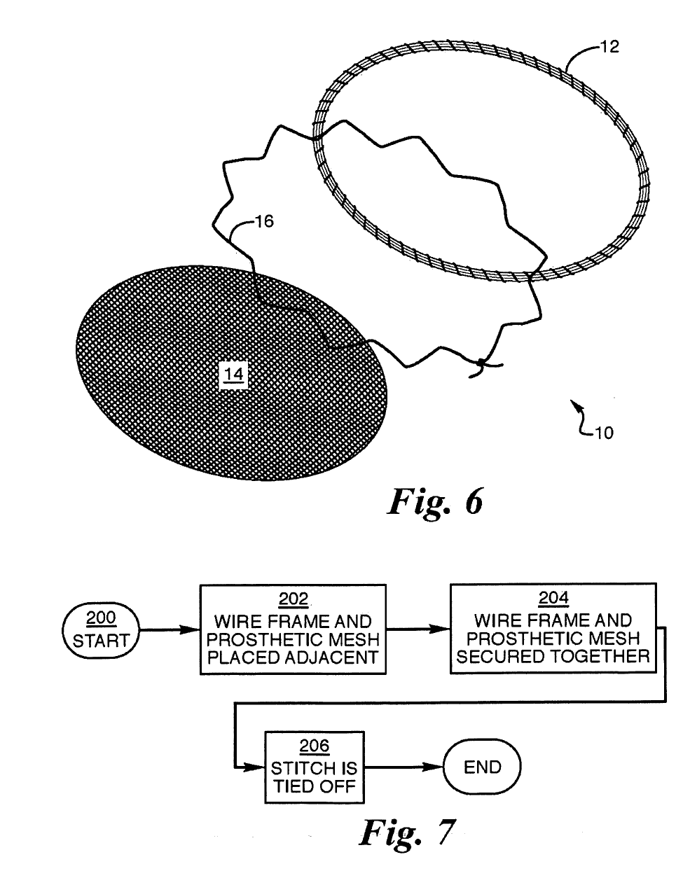

With reference to FIGS. 6 and 7, an exploded view and a process diagram are shown detailing the construction of a hernia patch in an embodiment of the present invention. After wire frame 12 is constructed, prosthetic fabric 14 and wire frame 12 are placed adjacent to one another at state 202 of process 200. Removable stitch 16 is then routed around wire frame 12 interlooping prosthetic fabric 14 so wire frame 12 and prosthetic fabric 14 are secured together at state 204. At state 206, removable stitch 16 is tied beginning end to end creating an endless loop.

Thus, embodiments of the PATCH FOR ENDOSCOPIC REPAIR OF HERNIAS are disclosed. One skilled in the art will appreciate the present teachings can be practiced with embodiments other than those disclosed. The disclosed embodiments are presented for purposes of illustration and not limitation, and the present teachings are limited only by the following claims.

We claim:

1. A hernia patch comprising:
(a) a frame formed from a shape memory alloy wherein the frame has an expanded shape when the shape memory alloy is in a austenite form and a rolled or folded compact shape when in a martensite form; and
(b) a prosthetic fabric material attached to the frame by a removable stitch.

2. The hernia patch of claim 1,
wherein the shape memory alloy comprises NiTi with a percentage of Ni in the alloy in a range of from 49 to 51 atomic percent.

3. The hernia patch of claim 2,
wherein the shape memory alloy exhibits a transformation temperature of about 37° C.

4. The hernia patch of claims 3,
wherein the prosthetic fabric material is selected from a group consisting of a woven mesh of polypropylene fibers, expanded PTFE, polyester, and other suitable materials approved for the repair of hernias.

5. The hernia patch as in claim 4,
wherein the prosthetic fabric material is generally planar and has a major longitudinal dimension in a range of from 8 to 40 cm and a minor transverse dimension in a range of from 6 to 30 cm.

6. A hernia patch for laparoscopic delivery comprising:
(a) a wire frame;
(b) a prosthetic fabric; and
(c) a removable stitch which couples the wire frame to the prosthetic fabric

7. The hernia patch of claim 6,
wherein the wire frame, prosthetic fabric, and removable stitch are arranged to be rolled up or folded for insertion through a tubular cannula into an abdominal space and when ejected from the cannula will assume the predetermined shape configuration.

8. The hernia patch of claim 7,
wherein the prosthetic fabric is polypropylene.

9. The hernia patch of claim 8,
wherein the prosthetic fabric is polytetrafluorethylene.

10. The hernia patch of claim 9,
wherein the prosthetic fabric is polyester.

11. The hernia patch of claim 10,
wherein the prosthetic fabric is a biomaterial.

12. A method of repairing a hernia, comprising the steps of:
(a) introducing a patch prosthesis into a patient;
(b) positioning the patch prosthesis over the hernia; and
(c) removing a wire frame from the patch prosthesis and the patient.

13. The method of claim 12,
further comprising the step of wrapping the patch prosthesis.

14. The method of claim 13,
further comprising the step of allowing the patch prosthesis to unwrap.

15. The method of claim 14,
further comprising the step of removing a removable stitch coupling the wire frame and a prosthetic fabric.

16. The method of claim 15,
further comprising the step of securing the prosthetic fabric to the patient.

17. A method of manufacturing a patch prosthetic, comprising the steps of:
(a) forming a wire frame;
(b) placing a prosthetic fabric adjacent to the wire frame; and
(c) securing the wire frame to the prosthetic fabric with a removable stitch.

18. The method of claim 17,
further comprising the step of tying off the removable stitch.

19. The method of claim 18,
wherein the wire frame is formed from a shape memory alloy wherein the wire frame has an expanded shape when the shape memory alloy is in an austenite form and a rolled or folded compact shape when in a martensite form.

20. The method of claim 19,
wherein the prosthetic fabric is selected from a group consisting of a woven mesh of polypropylene fibers, expanded PTFE, polyester fibers, and a suitable biomaterial.

21. The method of claim 20,
wherein the shape memory alloy comprises NiTi with a percentage of Ni in the alloy in a range of from 49 to 51 atomic percent.
